(19) United States
(12) Patent Application Publication
(10) Pub. No.: US 2024/0139215 A1
(43) Pub. Date: May 2, 2024

(54) CONTROLLED RELEASE FORMULATIONS OF HIGHLY LIPOPHILIC PHYSIOLOGICALLY ACTIVE SUBSTANCES

(71) Applicant: ADD Advanced Drug Delivery Technologies Ltd., Pratteln (CH)

(72) Inventors: Mirko Nowak, Lörrach (DE); Jay Jesko Nowak, Lörrach (DE); Annette Grave, Lörrach (DE); Monika Wentzlaff, Lörrach (DE); Sarah Barthold, Lörrach (DE); Christian Geugelin, Efringen-Kirchen (DE)

(73) Assignee: ADD Advanced Drug Delivery Technologies Ltd., Pratteln (CH)

(21) Appl. No.: 17/769,424

(22) PCT Filed: Oct. 16, 2020
(86) PCT No.: PCT/EP2020/079248

§ 371 Date: Apr. 15, 2022

(30) Foreign Application Priority Data
Oct. 16, 2019 (EP) 19203580.6

(51) Int. Cl. A61K 31/00; A61K 9/20; A61K 9/28

(52) U.S. Cl. CPC A61K 31/658; A61K 9/2054; A61K 9/2813; A61K 9/2866

(57) ABSTRACT

The invention relates to a solid dosage form that comprises a matrix with one or more highly lipophilic physiologically active substances, one or more water-soluble binders and, relative to the weight of all components, not more than 20 wt % of other auxiliary substances, wherein a physiologically active substance being highly lipophilic if it has a log P of 4 or more.

The present application claims priority from PCT Patent Application No. PCT/EP2020/079248 filed on Oct. 16, 2020, which claims priority from European Patent Application No. EP19203580.6 filed on Oct. 16, 2019, the disclosures of which are incorporated herein by reference in their entirety.

### FIELD OF THE INVENTION

The invention relates to formulations of highly lipophilic physiologically active substances, in particular controlled release formulations, as well as to their production. The highly lipophilic physiologically active substances are, for example, pharmaceutical active ingredients. An example of highly lipophilic pharmaceutical active ingredients are cannabinoids.

### BACKGROUND OF THE INVENTION

A number of physiologically active substances have highly lipophilic properties, i.e. they have a relatively high log P, for example a log P of 4 or more, the log P being the decimal logarithm of the n-octanol/water partition coefficient.

The provision of formulations, in particular oral formulations, with such physiologically active substances represents a particular challenge, in particular if a controlled release of the physiologically active substances is to be achieved.

Physiologically active substances with strong lipophilic properties include cannabinoids.

Cannabinoids are a heterogeneous group of pharmacologically active substances that have an affinity for the so-called cannabinoid receptors. The cannabinoids include, for example, tetrahydrocannabinol (THC) and the non-psychoactive cannabidiol (CBD).

Cannabinoids have raised considerable interest as drugs. There is evidence that cannabinoids can be beneficial for treating a number of clinical conditions, including pain, inflammation, epilepsy, sleep disorders, indication of multiple sclerosis, anorexia, and schizophrenia (N. Bruni et al., Cannabinoid Delivery Systems for Pain and Inflammation Treatment. Molecules 2018, 23, 2478).

However, the provision of suitable dosage forms is difficult because cannabinoids are highly lipophilic molecules (log P 6-7) with very low water solubility (2-10 μg/ml).

The low oral bioavailability of cannabinoids resulted in proposals of transdermal, intranasal and transmucosal administration.

In addition, due to the high lipophilicity of cannabinoids, salt formation (i.e. pH adjustment), cosolvency (e.g. ethanol, propylene glycol, PEG400), micellization (e.g. Polysorbate 80, Cremophor-ELP), emulsification including micro and nano emulsification, complexation (e.g. cyclodextrins) and encapsulation in lipid-based formulations (e.g. liposomes) are among the formulation strategies considered in the prior art. Nanoparticle systems have also been proposed (N. Bruni et al., Op. Cit.).

Various solid oral dosage forms have been proposed in the patent literature, for example in WO 2008/024490 A2 and in WO 2018/035030 A1. These documents do not contain data on release behaviour, so the practical suitability of the proposed forms for the administration of cannabinoids remains unclear.

WO 2015/065179 A1 describes compressed tablets which, in addition to cannabidiol, contain lactose and sucrose fatty acid monoesters.

Dronabinol (Δ9-THC) is marketed in the form of capsules (Marinol® and as an oral solution (Syndros®). The Marinol® capsules are soft gelatin capsules containing the active ingredient in sesame oil.

The finished drug Sativex® containing nabiximols is a mouth spray that is sprayed onto the inside of the cheek.

The recently approved preparation Epidiolex for the treatment of certain forms of epilepsy is provided in the form of an oral solution that in addition to the active ingredient cannabidiol contains the excipients absolute ethanol, sesame oil, strawberry aroma and sucralose.

Notwithstanding all of these proposals, however, there is still a need for improved dosage forms for highly lipophilic physiologically active substances, for example pharmaceutical active ingredients such as cannabinoids, in particular for solid oral dosage forms.

### SUMMARY OF THE INVENTION

An objective of the invention is to provide solid dosage forms, in particular solid oral dosage forms, for strongly lipophilic physiologically active substances, such as cannabinoids, which release the physiologically active substance/substances and which can be prepared in a simple manner.

This objective is achieved by providing a solid dosage form comprising a matrix with one or more highly lipophilic physiologically active substances; one or more water-soluble binders and not more than 20 wt.-%, based on the weight of all components, of further excipients, wherein a physiologically active substance is highly lipophilic if it has a log P of 4 or more.

Surprisingly, it was found that solid dosage forms, in particular solid oral dosage forms, of highly lipophilic physiologically active substances can be provided, wherein the release can be controlled with the help of the amount of water-soluble binder(s) relative to the amount of strongly lipophilic substance(s). The use of one or more water-soluble binders not only allows the formation of a matrix with the physiologically active substance(s), but also serves to control the release. In particular, a water-soluble binder promotes the release of the highly lipophilic substances that are only very slightly soluble in water. Only through the binder are these released in sufficient quantity and speed.

Further objectives and their solution can be concluded from the detailed description of the invention below.

### BRIEF DESCRIPTION OF THE FIGURES

With reference to the FIGURE the invention is explained in more detail below.

FIG. 1 shows the in vitro release from three pellet products comprising 2-[1R-3-methyl-6R-(1-methylethenyl)-2-cyclohexen-1-yl]-5-pentyl-1,3-benzenediol as active substance and low-viscosity hydroxypropylmethyl cellulose.

### DETAILED DESCRIPTION OF THE INVENTION

The dosage forms provided according to the invention contain one or more highly lipophilic physiologically active substances.

A substance is highly lipophilic if it has a log P of 4 or more. The log P is the decimal logarithm of the n-octanol/water partition coefficient. The partition coefficient can be determined experimentally. Values typically refer to room temperature (25° C.). The partition coefficient can also be roughly calculated from the molecular structure.

The pellets according to the invention are particularly suitable for physiologically active substances with a log P of 5 or more and especially for those with a log P of 6 or more.

The term “physiologically active substance” refers to a substance that is administered to a human or an animal in order to have an effect in the human or animal body. The physiologically active substance can, for example, be a pharmaceutical active substance of a human or veterinary medicinal product or a food supplement.

An example of highly lipophilic pharmaceutical active substances that can be used according to the invention are cannabinoids.

Cannabinoids can be both phytocannabinoids and synthetic cannabinoids.

Phytocannabinoids are a group of about 70 terpenophenolic compounds (V. R. Preedy (ed.), Handbook of Cannabis and Related Pathologies (1997)). These compounds typically contain a monoterpene residue that is attached to a phenolic ring and has a C₃-C₅ alkyl chain that is in the meta position to the phenolic hydroxyl group.

A preferred group of cannabinoids are tetrahydrocannabinols with the following general formula (1):

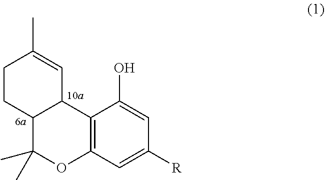

wherein R is selected from among C₁-C₂₀-alkyl, C₂-C₂₀-alkenyl or C₂-C₂₀-alkynyl, and optionally has one or more substituents.

In a further preferred group of compounds of the above general formula (1), R is selected from among C₁-C₁₀-alkyl or C₂-C₁₀-alkenyl, and optionally has one or more substituents.

In particular, in formula (1) R is an alkyl radical with the formula C₅H₁₁.

Compounds of general formula (1) can be present in the form of stereoisomers. The centres 6a and 10a preferably each have the R configuration.

The tetrahydrocannabinol is in particular Δ9-THC with the chemical name (6aR,10aR)-6,6,9-trimethyl-3-pentyl-6a, 7,8,10a-tetrahydro-6H-benzo[c]chromene-1-ol. The structure is reflected by the following formula (2):

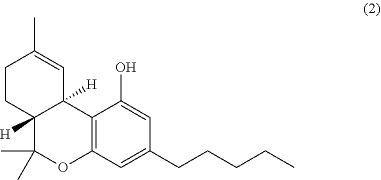 [SMILES: CCCCCc1cc(O)c2c(c1)OC(C)(C)[C@@H]1CCC(C)=C[C@@H]21]

Another preferred group of cannabinoids are cannabidiols with the following general formula (3):

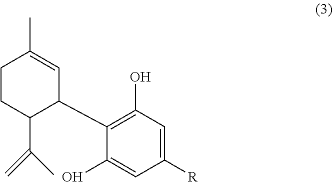

wherein R is selected from among C₁-C₂₀-alkyl, C₂-C₂₀-alkenyl or C₂-C₂₀-alkynyl, and optionally has one or more substituents.

In a further preferred group of compounds having the general formula (3) above, R is selected from among C₁-C₁₀-alkyl or C₂-C₁₀-alkenyl, and optionally has one or more substituents.

In particular, R in formula (3) is an alkyl radical with the formula C₅H₁₁.

The cannabidiol is in particular 2-[1R-3-methyl-6R-(1-methylethenyl)-2-cyclohexen-1-yl]-5-pentyl-1,3-benzenediol.

According to the invention, a combination of Δ9-THC ((6aR, 10aR)-6,6,9-trimethyl-3-pentyl-6a,7,8,10a-tetrahydro-6H-benzo[c]chromen-1-ol) and CBD (2-[1R-3-methyl-6R-(1-methylethenyl)-2-cyclohexen-1-yl]-5-pentyl-1,3-benzenediol) can be used.

Another preferred group of cannabinoids are cannabinols with the following general formula (4):

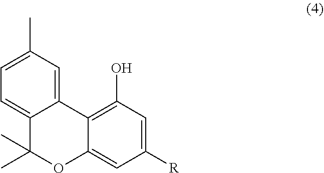

wherein R is selected from among C₁-C₂₀-alkyl, C₂-C₂₀-alkenyl or C₂-C₂₀-alkynyl, and optionally has one or more substituents.

In a further preferred group of compounds having the general formula (4) above, R is selected from among C₁-C₁₀-alkyl or C₂-C₁₀-alkenyl, and optionally has one or more substituents.

In particular, in formula (4) R is an alkyl radical having the formula C₅H₁₁.

The cannabinol is especially 6,6,9-trimethyl-3-pentyl-6H-dibenzo[b,d]pyran-1-ol.

According to the invention, cannabinoids or cannabinoid mixtures of hemp extracts can also be used.

For example, Nabiximols is a plant extract mixture used as a drug of the leaves and flowers of the hemp plant (Cannabis sativa L.) with standardized contents of tetrahydrocannabinol (THC) and cannabidiol (CBD).

Synthetic cannabinoids can also be used.

These include 3-(1,1-dimethylheptyl)-6,6a,7,8,10,10a-hexahydro-1-hydroxy-6,6-dimethyl-9H-dibenzo[b,d]pyran-9-one. This compound contains two stereogenic centres. The drug nabilone is a 1:1 mixture (racemate) of the (6aR,10aR) form and the (6aS,10aS) form. Nabilone is a preferred cannabinoid according to the invention.

Another example of a synthetic cannabinoid is JWH-018 (1-naphthyl-(1-pentylindol-3-yl)methanone.

According to the invention, one or more strongly lipophilic physiologically active substances, such as one or more pharmaceutical active ingredients, like cannabinoids, are contained in a matrix. The matrix preferably does not contain any other physiologically active substances.

The matrix contains one or more water-soluble binders. These binders are polymeric film-forming substances.

Examples of suitable water-soluble film formers are methyl cellulose (MC), hydroxypropyl methyl cellulose (HPMC), hydroxypropyl cellulose (HPC), hydroxyethyl cellulose (HEC), sodium carboxymethyl cellulose (Na-CMC) and polyvinyl pyrrolidone (PVP).

Hydroxypropylmethyl cellulose (HPMC), in particular low-viscosity HPMC, such as HPMC with a viscosity of a 2% (w/w) aqueous solution at 20° C. of 6 mPa·s or less is preferred.

An HPMC with a viscosity of a 2% (w/w) aqueous solution at 20° C. of 3 mPa·s, as is available under the trade name Pharmacoat® 603, is especially preferred.

The matrix of one or more highly lipophilic physiologically active substances and one or more water-soluble binders may contain other commonly used excipients, such as one or more fillers or carriers. According to the invention, the quantity of further excipients is limited to not more than 20 wt.-%, based on the weight of all components. Preferably, no more than 10 wt.-%, based on the weight of all components, of further excipients is comprised.

In a particularly preferred embodiment, the matrix consists of a highly lipophilic physiologically active substance/highly lipophilic physiologically active substances and binder(s), for example cannabinoid(s) and binder(s).

The matrix contains one or more water-soluble binders, based on the total amount of highly lipophilic physiologically active substances, in a total amount of 0.1-10 wt.-%, preferably in a total amount of 0.5-8 wt.-%, and in particular in a total proportion of 1-6 wt.-%.

It is assumed that if the amount of water-soluble binder is too small, the release takes place only very slowly and incompletely. By selecting the proportion in the specified ranges the release of the physiologically active substance can be adjusted. For example, the release from an oral dosage form can be adjusted so that the physiologically active substance is released over the conventional time of the gastrointestinal passage.

The solid oral dosage form according to the invention, comprising a matrix with one or more highly lipophilic physiologically active substances, may be provided and used in any form. For example, the dosage form may be provided in the form of granules, matrix pellets or matrix tablets, or may comprise any of these forms.

The preparation may be carried out in a manner known per se.

In a preferred embodiment, the dosage form contains matrix pellets.

The matrix pellets typically have a size in the range of 30 μm to 1800 μm, whereby the size can be determined by sieve analysis.

The matrix pellets may e.g. be offered in sachets, or they may be processed further.

For example, the matrix pellets may also be provided with one or more further coatings. This enables additional control of the release.

In a preferred embodiment, no coating controlling the release is provided.

The matrix pellets may also be used to obtain multiparticulate dosage forms. They can be filled into capsules or incorporated into tablets.

Matrix pellets with different release profiles may be combined in one dosage form (capsule/tablet/sachet).

The oral dosage forms according to the invention release the highly lipophilic physiologically active substance contained therein or, if more than one highly lipophilic physiologically active substance is contained, all the highly lipophilic physiologically active substances contained therein after ingestion in the digestive tract. The dosage forms are especially used for controlled release. They, in particular, release more than 30 wt.-% and less than 80 wt.-% of the physiologically active substance contained within two hours. In addition, they, especially, release more than 40 wt.-% and less than 90 wt.-% of the physiologically active substance contained within three hours. Furthermore, they release more than 50 wt.-% and less than 95 wt.-% of the physiologically active substance contained within four hours. If more than one physiologically active substance is comprised, the information relates to all substances contained.

In each case the release is determined in a blade stirrer apparatus in 1000 ml of phosphate buffer pH 6.8 with an addition of 0.4% Tween® 80 at 37° C.

### EXAMPLES

The examples below show how the release of a highly lipophilic physiologically active substance can be controlled by using a water-soluble film-forming substance.

### Example 1—Production of Pellets

Pellets were made using the quantities of ingredients shown in Table 1 below.

For this purpose 2 -[1R-3-methyl-6R-(1-methylethenyl)-2-cyclohexen-1-yl]-5-pentyl-1,3-benzenediol (Canapure PH) was dissolved in ethanol 96%. This active ingredient has a log P of about 6.1.

Another solution was prepared by dissolving HPMC (Pharmacoat® 603) in water.

The HPMC solution was then gradually added to the cannabidiol solution.

Then amorphous silicon dioxide (Syloid® 244 FP) was added.

It was stirred with a propeller stirrer.

The spray liquid obtained was sprayed onto starter cores made of microcrystalline cellulose (Cellets® 500).

This was done in a Mini-Glatt fluidized bed system with a Wurster insert. The air inlet air temperature was 40° C. The average spray rate was 0.5 g/min.

TABLE 1
Substances and quantities used

| | HPMC 0.8 | HPMC 0.6 | | HPMC 0.3 | |
|---|---|---|---|---|---|
| Formulation | Quantity | Quantity | | Quantity | |
| Solids | | | | | |
| Cellets 500 | 60.01 g/81.5% | 60.00 g/72.7% | | 60.00 g/72.7% | |
| Canapure PH | 21.02 g/16.1% | 21.00 g/24.2% | | 21.26 g/24.5% | |
| Pharmacoat 603 | 1.05 g/0.8% | 0.53 g/0.6% | | 0.26 g/0.3% | |
| Syloid 244 FP | 2.10 g/1.6% | 2.10 g/2.4% | | 2.10 g/2.4% | |
| Liquids (not included | | | | | |
| in the product) | | | | | |
| Ethanol 96% | 79.81 g | 79.83 | g | 79.82 | g |
| Pure water | 25.20 g | 25.21 | g | 25.21 | g |
| Spray liquid | | | | | |
| Solid content | 18.71% | 18.36% | | 18.36% | |
| (wt./wt.) | | | | | |
| Quantity sprayed | 72.80 g | 122.50 | g | 122.50 | g |

TABLE 2
Products

| Formulation | HPMC 0.8 | HPMC 0.6 | HPMC 0.3 |
|---|---|---|---|
| Theoretical | 73.63 g | 82.49 g | 82.49 g |
| yield | | | |
| Practical yield | 64.30 g/87.33% | 75.03 g/90.95% | 74.24 g/90.00% |
| Coating weight | 31.49% | 66.82% | 63.31% |
| gain | | | |

### Example 2—Release

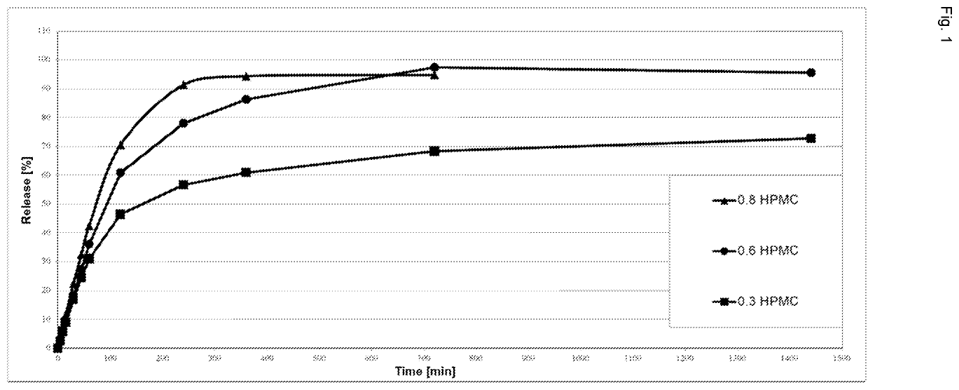

The release from the pellet products obtained in Example 1 is examined using a blade stirrer apparatus in 1000 ml phosphate buffer pH 6.8 with an addition of 0.4% Tween® 80, specifically at 37° C. The results obtained are shown in FIG. 1.

1. A solid dosage form comprising a matrix having one or more highly lipophilic physiologically active substances, one or more water-soluble binders and not more than 20 wt .- %, based on a weight of all components, other excipients, wherein a physiologically active substance being highly lipophilic if it has a log P of 4 or more.

2. The solid dosage form according to claim 1, wherein the one or more highly lipophilic physiologically active substances are one or more highly lipophilic pharmaceutically active ingredients.

3. the solid dosage form according to claim 2, wherein the one or more highly lipophilic pharmaceutically active ingredients are one or more cannabinoids.

4. The solid dosage form according to claim 3, wherein the one or more cannabinoids are selected from compounds of one of the following general formulas (1), (3) or (4): 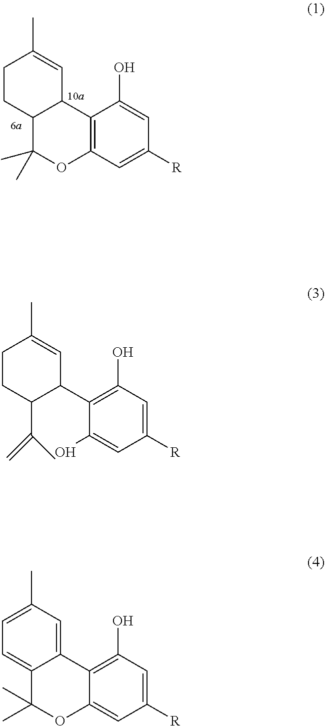
wherein R is, respectively, selected from among C₁-C₂₀-alkyl, C₂-C₂₀-alkenyl or C₂-C₂₀-alkynyl cannabinioids or cannabinoid mixtures of hemp extracts, such as nabiximols; and
synthetic cannabinoids, wherein the synthetic cannabinoids are one of nabilone or 1-naphthyl-(1-pentylindol-3-yl)methanone.

5. The solid dosage form according to claim 4, wherein the cannabinoid is 2-[1R-3-methyl-6R-(1-methylethenyl)-2-cyclohexen-1-yl]-5-pentyl-1,3-benzenediol; (6aR, 10aR)-6,6,9-trimethyl-3-pentyl-6a,7,8,10a-tetrahydro-6H-benzo[c]chromen-1-ol or a mixture thereof.

6. The solid dosage form according to claim 1, wherein the water-soluble binder is hydroxypropylmethyl cellulose (HPMC).

7. The solid dosage form according to claim 6, wherein the HPMC has a viscosity of 6 mPa·s or less in a 2% (w/w) aqueous solution at 20° C.

8. The solid dosage form according to claim 1, wherein the one or more binders are 0.3-10 wt.-% based on the total amount of highly liphilic physiologically active substance.

9. The solid dosage form according to claim 1, wherein the sold dosage form is provided in the form of at lest one of granules, matrix pellets or matrix tablets.

10. The solid dosage form according to claim 9, wherein the solid dosage form contains matrix pellets.

11. The solid dosage form according to claim 9, wherein the matrix pellets have a size in the range from 30 μm to 1800 μm.

12. The solid dosage form according to claim 1, wherein more than 30 wt.-% and less than 80 wt.-% of the one or more highly lipophilic physiologically active substances are released within two hours.

13. The solid dosage form according to claim 12, wherein more than 40 wt.-% and less than 90 wt.-% of the one or more highly lipophilic physiologically active substances are released within three hours.

14. The solid dosage form according to claim 13, wherein more than 50 wt.-% and less than 95 wt.-% of the one or more highly lipophilic physiologically active substances are released within four hours.
